(19) United States
(12) Patent Application Publication
(10) Pub. No.: US 2023/0398114 A1
(43) Pub. Date: Dec. 14, 2023

(54) METHODS OF TREATING CORONAVIRUS DISEASE AND COMPOUNDS FOR SAME

(71) Applicants: Kishor M. WASAN, Saskatoon (CA); Chris GALLIANO, New Orleans, LA (US); Supratik MUKHOPADHYAY, Baton Rouge, LA (US); Adam BESS, Baton Rouge, LA (US); Frej Knut Gosta BERGLIN, Baton Rouge, LA (US); Stephania CORMIER, Baton Rouge, LA (US); Allan ADER, New Orleans, LA (US); Nicholas GRIGGS, New Orleans, LA (US); Janet GOULD, New Orleans, LA (US); Tiffany CHO, New Orleans, LA (US); Julia ABRAMOV, New Orleans, LA (US); Peter HNIK, New Orleans, LA (US); Michal BRYLINSKI, Baton Rouge, LA (US); SKYMOUNT MEDICAL US INC., New Orleans, LA (US); THE BOARD OF SUPERVISORS OF LOUISIANA STATE UNIVERSITY AND AGRICULTURAL AND MECHANICAL COLLEGE, Baton Rouge, LA (US)

(72) Inventors: Kishor M. WASAN, Saskatoon (CA); Chris GALLIANO, New Orleans, LA (US); Supratik MUKHOPADHYAY, Baton Rouge, LA (US); Adam BESS, Baton Rouge, LA (US); Frej Knut Gosta BERGLIND, Baton Rouge, LA (US); Stephania CORMIER, Baton Rouge, LA (US); Allan ADER, New Orleans, LA (US); Nicholas GRIGGS, New Orleans, LA (US); Janet GOULD, New Orleans, LA (US); Tiffany CHO, New Orleans, LA (US); Julia ABRAMOV, New Orleans, LA (US); Peter HNIK, New Orleans, LA (US); Michal BRYLINSKI, Baton Rouge, LA (US)

(21) Appl. No.: 18/035,011

(22) PCT Filed: Nov. 2, 2021
(86) PCT No.: PCT/US2021/057754

§ 371 Date: May 2, 2023

Related U.S. Application Data
(60) Provisional application No. 63/168,766, filed on Mar. 31, 2021.
(60) Provisional application No. 63/165,510, filed on Mar. 24, 2021.

(30) Foreign Application Priority Data
Nov. 2, 2020 (CA) 3,097,717

(51) Int. Cl. A61K 31/506; A61K 31/4184; A61P 31/14

(52) U.S. Cl. CPC A61K 31/506; A61K 31/4184; A61P 31/14

(57) ABSTRACT

A tyrosine kinase inhibitor for use in the treatment of COVID-19 and/or its associated symptoms. Also disclosed is a method of treating an individual infected with a coronavirus, wherein said method comprises the steps of: providing a tyrosine kinase inhibitor; and administering said tyrosine kinase inhibitor to said individual in a dosage amount sufficient to prevent/stabilize/reduce the risks and/or symptoms associated with a coronavirus infection.

### TECHNICAL FIELD

This disclosure relates to compounds and compositions for use in the treatment of coronavirus diseases. More particularly, the embodiments of the present disclosure relate to compounds and compositions capable of targeting a mode of interaction between a coronavirus and subject cells.

### BACKGROUND

The appearance of a novel coronavirus, referred to as SARS-CoV-2, on the world stage has affected substantially every population in the world. This virus has afflicted millions of individuals and caused a disease, referred to as COVID-19. COVID-19 can develop into a significant health risk and result in death, which has placed a high strain on healthcare resources and society in general.

SARS-CoV-2 is a single-strand, positive-sense ribonucleic acid (RNA) virus with a similar receptor-binding domain structure to that of SARS-CoV and MERS-CoV. SARS-CoV-2 is transmitted between individuals via airborne droplets accessing nasal mucosa. Within the nasal mucosa SARS-CoV-2 can rapidly reproduce and be shed in nasal secretions (sputum). Sputum can be transmitted to other individuals via airborne droplets, thus repeating the transmission cycle. The SARS-CoV-2 virus can spread between individuals before the onset of symptoms, during the symptomatic period and even after recovery.

The clinical spectrum of the infection is wide, ranging from mild signs of an upper respiratory tract infection to severe pneumonia, multi-organ failure and death. At the onset, SARS-CoV-2 primarily attacks the respiratory system, as it represents the main point of entry into the host, but SARS-CoV-2 also can affect multiple organs of an infected individual. The severity of COVID-19 is typically associated with comorbidities such as, but not limited to: hypertension, diabetes, obesity, cardiovascular disease, and/or advanced age that can exacerbate the consequences of COVID-19.

There exists a need for a therapy that is capable of mitigating the impact of COVID-19 in such a manner that it slows down physiological impact on an infected individual.

### SUMMARY

The embodiments of the present disclosure provide one or more therapies for ameliorating and/or inhibiting some or substantially all of the risks, symptoms and development of severe disease in a subject infected with a coronavirus. In some embodiments of the present disclosure, the coronavirus is SARS-CoV-2.

Some embodiments of the present disclosure relate to a use of a tyrosine kinase inhibitor for ameliorating and/or inhibiting some or substantially all of the risks, symptoms and development of severe disease caused by a coronavirus infection.

Some embodiments of the present disclosure relate to a use of a first tyrosine kinase inhibitor and a second tyrosine kinase inhibitor for ameliorating and/or inhibiting some or substantially all of the risks, symptoms or development of severe disease caused by a coronavirus infection.

Some embodiments of the present disclosure relate to a method of treating an individual exposed to or infected with a coronavirus, wherein said method comprises the steps of: providing a therapeutically effective amount of a tyrosine kinase inhibitor; and, administering the therapeutically effective amount of the tyrosine kinase inhibitor to said individual to ameliorate and/or inhibit some or substantially all of the risks, symptoms and development of severe disease in a subject infected with a coronavirus.

Some embodiments of the present disclosure relate to a method of treating an individual exposed to or infected with a coronavirus, wherein said method comprises the steps of: providing a therapeutically effective amount of a first tyrosine kinase inhibitor and a second therapeutically effective amount of a second tyrosine kinase inhibitor; administering said therapeutically effective amounts of the first and second tyrosine kinase inhibitors to said individual for ameliorating and/or inhibiting some or substantially all of the risks, symptoms or development of severe disease caused by a coronavirus infection.

Some embodiments of the present disclosure relate to a method of making an agent/target virion complex, the method comprising a step of administering a therapeutically effective amount of the agent to a subject, wherein the agent/target virion complex inhibits the agent/target virion complex from entering a subject's cell, fusing with a subject's cell and/or replicating within a subject's cell. In some embodiments of the present disclosure, the agent is a tyrosine kinase inhibitor or two or more tyrosine kinase inhibitors.

Some embodiments of the present disclosure relate to a method of making an agent/target cell complex, the method comprising a step of administering a therapeutically effective amount of the agent to a subject, wherein the agent/target cell complex inhibits a coronavirus from: entering into the agent/target cell complex, fusing with the agent/target cell complex and/or replicating within the agent/target cell complex. In some embodiments of the present disclosure, the agent is a tyrosine kinase inhibitor or two or more tyrosine kinase inhibitors.

Some embodiments of the present disclosure relate to a pharmaceutical composition that comprises a first tyrosine kinase inhibitor in a first therapeutically effective amount; a second tyrosine kinase inhibitor in a second therapeutically effective amount, wherein the first and second therapeutically effective amounts are different or not; and at least one excipient.

In the embodiments of the present disclosure, the tyrosine kinase inhibitor and the second tyrosine kinase inhibitor are each one of: Axitinib; Mebendazole; Crizotinib; Bosutinib; Vandetanib; Midostaurin; Gefitinib; Niclosamide; Imatinib; Dabrafenib; Entrectinib; Sorafenib; Dacomitinib; Sunitinib; Alectinib; Baricitinib; or Ibrutinib.

In some embodiments of the present disclosure, the tyrosine kinase inhibitor or the second tyrosine kinase inhibitor may be: Axitinib. In some embodiments of the present disclosure, the tyrosine kinase inhibitor or the second tyrosine kinase inhibitor may be: Mebendazole. In some embodiments of the present disclosure, the tyrosine kinase inhibitor or the second tyrosine kinase inhibitor may be: Crizotinib. In some embodiments of the present disclosure, the tyrosine kinase inhibitor or the second tyrosine kinase inhibitor may be: Bosutinib. In some embodiments of the present disclosure, the tyrosine kinase inhibitor or the second tyrosine kinase inhibitor may be: Vandetanib. In some embodiments of the present disclosure, the tyrosine kinase inhibitor or the second tyrosine kinase inhibitor may be: Midostaurin. In some embodiments of the present disclosure, the tyrosine kinase inhibitor or the second tyrosine kinase inhibitor may be: Gefitinib. In some embodiments of the present disclosure, the tyrosine kinase inhibitor or the second tyrosine kinase inhibitor may be: Niclosamide. In some embodiments of the present disclosure, the tyrosine kinase inhibitor or the second tyrosine kinase inhibitor may be: Imatinib. In some embodiments of the present disclosure, the tyrosine kinase inhibitor or the second tyrosine kinase inhibitor may be: Dabrafenib. In some embodiments of the present disclosure, the tyrosine kinase inhibitor or the second tyrosine kinase inhibitor may be: Entrectinib. In some embodiments of the present disclosure, the tyrosine kinase inhibitor or the second tyrosine kinase inhibitor may be: Sorafenib. In some embodiments of the present disclosure, the tyrosine kinase inhibitor or the second tyrosine kinase inhibitor may be: Dacomitinib. In some embodiments of the present disclosure, the tyrosine kinase inhibitor or the second tyrosine kinase inhibitor may be: Sunitinib. In some embodiments of the present disclosure, the tyrosine kinase inhibitor or the second tyrosine kinase inhibitor may be: Alectinib. In some embodiments of the present disclosure, the tyrosine kinase inhibitor or the second tyrosine kinase inhibitor may be: Baricitinib. In some embodiments of the present disclosure, the tyrosine kinase inhibitor or the second tyrosine kinase inhibitor may be: Ibrutinib.

Without being bound by any particular theory, it is postulated that tyrosine kinase inhibitors may target the SARS-CoV-2 virus by inhibiting the entry or fusion of the virus with a subject's cell and/or inhibiting replication of the virus once inside a subject's cell. While Mebendazole is a known anthelminthic, surprisingly it may also be useful in treating COVID-19 because Mebendazole interferes in viral tubulin formation and it may target viral calmodulin-domain protein kinase 1 also.

Without being bound by any particular theory, it is postulated that a tyrosine kinase inhibitor, such as Imatinib, when used alone or in combination with Mebendazole, may also be useful as a therapy for ameliorating and/or inhibiting some or substantially all of the risks, symptoms and development of severe disease in a subject infected with a coronavirus. In some embodiments of the present disclosure, Imatinib which is a known ABL kinase inhibitor, may be useful alone or as part of a combination treatment, with Mebendazole, for COVID-19 because Imatinib is known to target one or more of TK ALK, platelet-derived growth factor receptor alpha, TK ABL1/Bcr-Abl, or Mast/stem cell growth factor receptor Kit.

### BRIEF DESCRIPTION OF THE DRAWINGS

These and other features of the present disclosure will become more apparent in the following detailed description in which reference is made to the appended drawings.

FIG. 1 is a line graph that represents in vitro data from samples, obtained according to embodiments of the present disclosure, regarding efficacy of viral inhibition and toxicity of a tyrosine kinase inhibitor, used according to embodiments of the present disclosure.

FIG. 2 shows two histograms of in vitro data from samples, obtained according to embodiments of the present disclosure, regarding use of a second tyrosine kinase inhibitor, wherein FIG. 2A shows TCID50 for various concentrations of the second tyrosine kinase inhibitor; and, FIG. 2B shows viral inhibition for various concentrations of the second tyrosine kinase inhibitor.

FIG. 3 shows a line graph of in vitro data from samples, obtained according to embodiments of the present disclosure, regarding cell viability for various concentrations of the second tyrosine kinase inhibitor.

FIG. 4 shows a scatter plot of in vitro data from samples, obtained according to embodiments of the present disclosure, regarding viral inhibition and cell viability for various concentrations of the second tyrosine kinase inhibitor at four concentrations of the tyrosine kinase inhibitor.

FIG. 5 shows a line graph of in vivo data from experimental samples, obtained according to embodiments of the present disclosure, regarding changes in body weight following exposure to a coronavirus and treatment with a placebo or various tyrosine kinase inhibitors, wherein FIG. 5A shows all treatment groups; and, FIG. 5B shows a placebo group and three treatment groups.

FIG. 6 is two histograms that shows levels of ALT, AST and BUN detected in experimental samples, obtained according to embodiments of the present disclosure, wherein FIG. 6 A shows data from six experimental groups (placebo and 5 treatment groups); and, FIG. 6B shows data from the placebo group and the same three treatment groups of FIG. 5B.

FIG. 7 shows two scatter plots representing data obtained from the six experimental groups in FIG. 5, wherein FIG. 7A shows normalized mRNA levels; and, FIG. 7B shows virus titer levels.

FIG. 8 shows two line graphs of in vitro data from samples, obtained according to embodiments of the present disclosure, wherein FIG. 8A shows cell viability for various concentrations of another tyrosine kinase inhibitor; and FIG. 8B shows reduction in viral growth for various concentrations of the same tyrosine kinase inhibitor of FIG. 8A.

### DETAILED DESCRIPTION

Unless defined otherwise, all technical and scientific terms used herein have the meanings that would be commonly understood by one of skill in the art in the context of the present description. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, preferably within about 15%, preferably within about 10%, preferably within about 5% of a given value or range. It is understood that such a variation is always included in any given value provided herein, whether or not it is specifically referred to.

As used herein, the term “activity” is used interchangeably with the term “functionality” and both terms refer to the physiologic action of a biomolecule.

As used herein, the terms “agent” and “therapeutic agent” refer to a substance that, when administered to a subject, causes one or more chemical reactions and/or one or more physical reactions and/or or one or more physiological reactions and/or one or more pharmacological reactions and/or one or more immunological reactions in the subject.

As used herein, the term “ameliorate” refers to improve and/or to make better and/or to make more satisfactory.

As used herein, the term “cell” refers to a single cell as well as a plurality of cells or a population of the same cell type or different cell types. Administering an agent to a cell includes in vivo, in vitro and ex vivo administrations and/or combinations thereof.

As used herein, the term “complex” refers to an association, either direct or indirect, between one or more particles of an agent and one or more target cells or target virions. This association results in a change in the metabolism or functionality of the target cells or target virions. As used herein, the phrase “change in metabolism” refers to an increase or a decrease in the one or more of the targets' production of one or more proteins, and/or any post-translational modifications of one or more proteins. As used herein, the phrase “change in functionality” refers to a difference in physiological function of one or more aspects of the target within an agent/target complex as compared to a target that is not part of such a complex.

As used herein, the terms “dysregulation” and “dysregulated” refer to situations or conditions wherein homeostatic control systems have been disturbed and/or compromised so that one or more metabolic, physiologic and/or biochemical systems within a subject operate partially or entirely without said homeostatic control systems.

As used herein, the term “excipient” refers to any substance, not itself an agent, which may be used in a composition for delivery of one or more agents, and the like to a subject or alternatively combined with . . . one or more carriers and the like (e.g., to create a pharmaceutical composition) to improve its handling or storage properties or to permit or facilitate formation of a dose unit of the composition (e.g., formation of a topical hydrogel which may then be optionally incorporated into a transdermal patch). Excipients include, by way of illustration and not limitation, binders, disintegrants, taste enhancers, solvents, thickening or gelling agents (and any neutralizing agents, if necessary), penetration enhancers, solubilizing agents, wetting agents, antioxidants, lubricants, emollients, substances added to mask or counteract a disagreeable odor, fragrances or taste, substances added to improve appearance or texture of the composition and substances used to form the pharmaceutical compositions. Any such excipients can be used in any dosage forms according to the present disclosure. The foregoing classes of excipients are not meant to be exhaustive but merely illustrative.

As used herein, the terms “inhibit”, “inhibiting”, and “inhibition” refer to a decrease in activity, response, or other biological parameter of a biologic process, disease, disorder or symptom thereof. This can include but is not limited to the complete ablation of the activity, response, condition, or disease. This may also include, for example, a 10% reduction in the activity, response, condition, or disease as compared to the native or control level. Thus, the reduction can be a 10%, 20%, 30%, 40%, 50%, 60%, 70%, 80%, 90%, 100%, or any amount of reduction in between the specifically recited percentages, as compared to native or control levels.

As used herein, the term “medicament” refers to a medicine and/or pharmaceutical composition that comprises an agent and that can promote recovery from a disease, disorder or symptom thereof and/or that can prevent a disease, disorder or symptom thereof and/or that can inhibit the progression of a disease, disorder, or symptom thereof.

As used herein, the term “pharmaceutical composition” means any composition comprising, but not necessarily limited to, one or more agents to be administered a subject in need of therapy or treatment of a disease, disorder or symptom thereof. Pharmaceutical compositions may include additives such as pharmaceutically acceptable carriers, pharmaceutically accepted salts, excipients and the like. Pharmaceutical compositions may also additionally include one or more further active ingredients such as antimicrobial agents, anti-inflammatory agents, anaesthetics, analgesics, and the like.

As used herein, the term “pharmaceutically acceptable carrier” refers to an essentially chemically inert and nontoxic component within a pharmaceutical composition or medicament that does not inhibit the effectiveness and/or safety of the one or more agents. Some examples of pharmaceutically acceptable carriers and their formulations are described in Remington (1995, The Science and Practice of Pharmacy (19th ed.) ed. A. R. Gennaro, Mack Publishing Company, Easton, PA), the disclosure of which is incorporated herein by reference. Typically, an appropriate amount of a pharmaceutically acceptable carrier is used in the formulation to render said formulation isotonic. Examples of suitable pharmaceutically acceptable carriers include, but are not limited to: saline solutions, glycerol solutions, ethanol, N-(1(2, 3-dioleyloxy)propyl)-N,N,N-trimethylammonium chloride (DOTMA), dioleolphosphotidylethanolamine (DOPE), and liposomes. Such pharmaceutical compositions contain a therapeutically effective amount of the agent, together with a suitable amount of one or more pharmaceutically acceptable carriers and/or excipients so as to provide a form suitable for proper administration to the subject. The formulation suits the route of administration. For example, oral administration may require enteric coatings to protect the agent from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the phrases “prevent”, “prevention of” and “preventing” refer to avoiding the onset or progression of a disease, disorder, or a symptom thereof.

As used herein, the term “subject” refers to any therapeutic target that receives the agent. The subject can be a vertebrate, for example, a mammal including a human. The term “subject” does not denote a particular age or sex. The term “subject” also refers to one or more cells of an organism, an in vitro culture of one or more tissue types, an in vitro culture of one or more cell types, ex vivo preparations, and/or a sample of biological materials such as tissue and/or biological fluids.

As used herein, the term “target cell” refers to one or more cell types within a subject that can interact with a coronavirus by the virus fusing with the outer membrane of the one or more cell types, entering into the cell and/or replicating therein. Without being bound to any particular theory, target cells of a subject can include any cells within a subject that express the receptors and/or co-factors required for viral interaction. Examples of these types of cells include, but are not limited to: epithelial cells of the upper airways and conducting airways (ciliated and non-ciliated); alveolar epithelial cells (both type 1 and 2); epithelial cells and neurons of the olfactory system; neurons of the central or peripheral nervous system; epithelial cells, enterocytes and gland cells of the gastrointestinal tract; cells of the blood, including immune effector cells; cardiovascular cells, and renal cells.

As used herein, the term “target virion” refers to one or more viral particles of coronavirus that have the capacity to cause a viral infection within a target cell. In some embodiments of the present disclosure, the viral particles are of one or more variants of SARS-CoV-2.

As used herein, the term “therapeutically effective amount” refers to the amount of the agent used that is of sufficient quantity to ameliorate, prevent, treat and/or inhibit one or more of a disease, disorder or a symptom thereof. The “therapeutically effective amount” will vary depending on the agent used, the route of administration of the agent and the severity of the disease, disorder or symptom thereof. The subject's age, weight and genetic make-up may also influence the amount of the agent that will be a therapeutically effective amount.

As used herein, the terms “treat”, “treatment” and “treating” refer to obtaining a desired pharmacologic and/or physiologic effect. The effect may be prophylactic in terms of completely or partially preventing an occurrence of a disease, disorder or symptom thereof and/or the effect may be therapeutic in providing a partial or complete amelioration or inhibition of a disease, disorder, or symptom thereof. Additionally, the term “treatment” refers to any treatment of a disease, disorder, or symptom thereof in a subject and includes: (a) preventing the disease from occurring in a subject which may be predisposed to the disease but has not yet been diagnosed as having it; (b) inhibiting the disease, i.e., arresting its development; and, (c) ameliorating the disease.

As used herein, the terms “unit dosage form” and “unit dose” refer to a physically discrete unit that is suitable as a unitary dose for patients. Each unit contains a predetermined quantity of the agent and optionally, one or more suitable pharmaceutically acceptable carriers, one or more excipients, one or more additional active ingredients, or combinations thereof. The amount of agent within each unit is a therapeutically effective amount.

In embodiments of the present disclosure, the pharmaceutical compositions disclosed herein comprise one or more agents as described above in a total amount by weight of the composition of about 0.1% to about 95%. For example, the amount of the agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%. about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95% or more.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also, encompassed within the disclosure, subject to any specifically excluded limit in the stated range. Where the stated range includes one or both of the limits, ranges excluding either or both of those included limits are also included in the disclosure.

The appearance of COVID-19 on the world stage has affected every population in the world, infecting millions of individuals, a number which continues to increase.

The protein tyrosine kinases (PTK) are one of the most important enzyme families involved in cell signal transduction. PTKs facilitate the transfer of adenosine triphosphate (ATP)-γ-phosphate to tyrosine residues of the substrate protein, and thus directly impacting a number of physiological and biochemical processes including but not limited to phosphorylation, regulating cell growth, differentiation, and cell death. This covalent post-translational modification is a pivotal component of normal cellular communication and maintenance of homeostasis. PTKs are implicated in several steps of neoplastic development and progression. PTK signaling pathways normally prevent deregulated proliferation or contribute to sensitivity towards apoptotic stimuli.

PTKs are important mediators of the signaling cascade, determining key roles in diverse biological processes like growth, differentiation, metabolism and apoptosis in response to external and internal stimuli. It has been determined that tyrosine kinase have an important role in the pathophysiology of cancer. Though their activity is typically regulated in normal cells, they may acquire transforming functions due to mutation(s), overexpression and autocrine paracrine stimulation, leading to malignancy.

These signaling pathways are often genetically or epigenetically altered in cancer cells to impart a selection advantage to the cancer cells. Tyrosine kinase inhibitors have proven to be valuable therapeutics when there is an abnormal expression of PTK.

In light of the state of the art, potential applications of the use of tyrosine kinase inhibitors to provide some therapeutic benefits when dealing with a coronavirus, such as SARS-CoV-2 were identified. Given the haste and the magnitude of the pandemic, it may be advantageous to be identify compounds and compositions that are already approved for therapeutic use.

COVID-19 patients have demonstrated that the time from symptom onset to development of dyspnea may be between 5 to 10 days. In some COVID-19 patients, it may take between 10 and 14 days to develop severe respiratory distress syndrome. The probability of progress to end-stage disease is unpredictable, with the majority of these patients dying from multi-organ failure. Inhibiting progression in spontaneously breathing patients with mild to moderate COVID-19 could translate to reduced morbidity and mortality and in a lower use of limited healthcare resources.

The SARS-CoV-2 virus may cause gastrointestinal symptoms, such as vomiting, diarrhea, or abdominal pain during the early phases of the disease. Gastrointestinal dysfunction may induce changes in intestinal microbes and an increase in inflammatory cytokines.

According to the embodiments of the present disclosure, tyrosine kinase inhibitors may be used in the treatment of COVID-19 and/or symptoms thereof and/or prophylaxis to avoid or mitigate infection following exposure to the SARS-CoV-2 virus.

Some embodiments of the present disclosure relate to a method of treating a subject that has been exposed to the SARS-CoV-2 virus. The steps of the method include providing a therapeutically effective amount of a tyrosine kinase inhibitor and administering that therapeutically effective amount to the subject. The therapeutically effective amount of the tyrosine kinase inhibitor may ameliorate or inhibit some or substantially all of the symptoms of a coronavirus related disease. While the SARS-CoV-2 virus and the related disease of COVID-19 are provided as examples of a coronavirus and a related disease, those skilled in the art will appreciate that the present disclosure is not limited to just SARS-CoV-2 and COVID-19—other coronaviruses and relates diseases are contemplated herein.

Some embodiments of the present disclosure relate to a method of treating a subject by administering a second therapeutically effective amount of a second tyrosine kinase inhibitor to the subject.

Some embodiments of the present disclosure relate to a method of forming a complex between an agent and a target cell. The method includes the steps of administering a therapeutically effective amount of the agent to the target cell and forming the target cell/agent complex. The complex may be inhibit fusion of the coronavirus with the target cell. The complex may inhibit entry of coronavirus particles into the target cell. The complex may inhibit replication of the coronavirus within the target cell. The agent and the second agent both comprise a tyrosine kinase inhibitor.

Some embodiments of the present disclosure relate to administering a therapeutically effective dose of an agent to form a target cell/agent complex and administering a second therapeutically effective dose of a second agent to form a second target cell/agent complex. The target cell of the first complex may be the same or a different target cell than the second target cell.

Some embodiments of the present disclosure relate to a use of a tyrosine kinase inhibitor for treating a subject who has been exposed to or infected with a coronavirus. In some embodiments of the present disclosure, the use may further include using a second tyrosine kinase inhibitor as part of the same treatment.

Some embodiments of the present disclosure relate to a pharmaceutical composition useful for treating a subject who has been exposed to or infected with a coronavirus. The pharmaceutical composition comprising a therapeutically effective amount of an agent and one or more excipients. In some embodiments of the present disclosure, the pharmaceutical composition may comprise a first agent and a second agent, where both agents comprise a different tyrosine kinase inhibitor.

According to some embodiments of the present disclosure, the tyrosine kinase inhibitors may be one of Axitinib; Mebendazole; Crizotinib; Bosutinib; Vandetanib; Midostaurin; Gefitinib; Niclosamide; Imatinib; Dabrafenib; Entrectinib; Sorafenib; Dacomitinib; Sunitinib; Alectinib; Baricitinib; or Ibrutinib.

According to some embodiments of the present disclosure, Axitinib, which is a tyrosine kinase inhibitor, may be useful in treating COVID-19 because of its actions as a tyrosine kinase (TK) inhibitor and/or as Serine/Threonine protein kinase (STK) inhibitor. Axitinib is known to target vascular Endothelial Growth Factor (VEGF) receptor 1 (0.2 nM), Tyrosine Kinase (TK) ABL1 (2.6 nM), Serine/Threonine protein kinase (STK) PLK4 (43 nM), TBK1 (470 nM).

According to some embodiments of the present disclosure, Mebendazole, may be useful in treating COVID-19 because of its actions as a tyrosine kinase inhibitor and/or its actions in interfering in tubulin formation. Mebendazole is known to interfere in tubulin formation as well as Calmodulin-domain protein kinase 1 (670 nM), VEGF1 (3.6 μM), and TK ABL1 (5 μM). Mebendazole is an anthelmintic agent that exhibits broad spectrum activity against single or mixed helminthic infestations. Mebendazole binds selectively to tubulin in the intestines of helminths and interferes with microtubule formation, blocking glucose uptake and generation of ATP, resulting in impaired digestive function, inhibition of larval development, and death of the helminths. Preclinical studies suggest that Mebendazole may have anti-neoplastic activity by inhibiting a wide range of factors involved in tumor progression such as tubulin polymerization, angiogenesis, matrix metalloproteinases, and multi-drug resistance protein transporters. Mebendazole was also shown to inhibit several drug transporters including ATP-binding cassette (ABC) transporters.

The SARS-CoV-2 virus has spike proteins that are known to interact with cytoskeleton filaments (e.g., actin, tubulin) for successful entry of the virus into host cells. Entry of the virus is a key step in viral infection. However, in a study evaluating human coronavirus NL63, actin inhibitors reduced virus entry, but the microtubule inhibitor, nocodazole, did not.

Mebendazole effectively reduced viral replication in in vitro studies at doses similar to or lower than anthelmintic therapeutic amounts. For example, in Vero E6 nonhuman primate cells that were infected with SARS-CoV-2, an IC50 range of 0.25 to 1.2 μM was determined for Mebendazole. These concentrations fall below peak plasma concentrations (1.69 μM; 0.5 μg/mL) that were reported in patients undergoing chronic Mebendazole therapy. However, compared to the Vero E6 cells, the antiviral activity of Mebendazole in a human hepatoma cell line (Huh 7.5 cells) infected with SARS-CoV-2 appeared to be lower.

Following oral administration of Mebendazole, peak plasma concentrations are achieved in 2 to 4 hours. Animal and human studies indicate slight to moderate oral absorption. In humans, less than about 10% of a single oral dose reaches systemic circulation due to extensive first-pass metabolism. An oral bioavailability of 17% has been reported. Following administration of 100 mg twice daily for three consecutive days, plasma concentrations of Mebendazole did not exceed 0.03 μg/mL (0.1 μM). Following long-term oral administration, increased plasma concentrations resulted in about a 3-fold higher exposure to steady-state. In subjects on chronic Mebendazole chemotherapy, peak plasma concentrations ranged from 0.1 to 0.5 μg/mL (0.3 to 1.69 μM). Systemic exposure is reportedly higher in children compared to adults. Evidence suggests small amounts of Mebendazole are present in human milk following oral administration. Mebendazole is extensively metabolized in the liver to several inactive metabolites that demonstrate higher plasma concentrations compared to those of Mebendazole. The metabolites of Mebendazole likely undergo enterohepatic recirculation. Excretion is primarily fecal; <2% of an oral dose is excreted in urine. The elimination half-life ranges from 3 to 6 hours.

According to some embodiments of the present disclosure, Crizotinib, which is an ALK kinase inhibitor, may be useful in treating COVID-19 because of its actions as a Tyrosine kinase inhibitor. Crizotinib is known to target MutT homolog 1 protein (330 nM).

According to some embodiments of the present disclosure, Bosutinib, which is an ABL/SRC kinase inhibitor, may be useful in treating COVID-19 because of its actions as a Tyrosine kinase inhibitor. Bosutinib is known to target proto-oncogene TK Src (1.1 nM).

According to some embodiments of the present disclosure, Vandetanib, which is a tyrosine kinase inhibitor, may be useful in treating COVID-19 because of its actions as an EGFR inhibitor where it can prevent excessive fibrotic response and impact viral entry. Vandetanib is known to target EGFR (11 nM), VEGF2 (15 nM), Proto-oncogene TK Ret (44 nM) TK ABL1 (86 nM), TK Src (186 nM), platelet-derived growth factor receptor beta (477 nM), angiopoietin-1 receptor (567 nM).

According to some embodiments of the present disclosure, Midostaurin, which is a tyrosine kinase inhibitor, may be useful in treating COVID-19 because of its actions as a tyrosine kinase inhibitor. Midostaurin is known to target Proto-oncogene TK Src (800 nM) and EGFR (1900 nM).

According to embodiments of the present disclosure, Gefitinib, which is a tyrosine kinase inhibitor, may be useful in treating COVID-19 because of its actions as a tyrosine kinase inhibitor, EGFR inhibitor—where it can prevent excessive fibrotic response and impact viral entry. Gefitinib is known to target EGFR (0.1 nM), STK RIPK2 (3.8 nM), and TK erbB-4 (7.6 nM).

According to embodiments of the present disclosure, Niclosamide, which is also an anthelminthic, may be useful in treating COVID-19 because of its actions as an E3 ligase S-Phase kinase associated protein 2 (SKP2) inhibitor, Tyrosine kinase inhibitor STAT3. Niclosamide is known to target STAT3 (250 nM), Proto-oncogene TK Src (1 μM), TK JAK2 (1 μM), EGFR (1 μM), Fibroblast growth factor receptor (1 μM), and VEGF2 (2 μM).

According to some embodiments of the present disclosure, Imatinib, which is an ABL kinase inhibitor, would be useful in treating COVID-19 because of its actions as a tyrosine kinase inhibitor. Imatinib is known to target TK ALK (1 nM), platelet-derived growth factor receptor alpha (2 nM), TK ABL1/Bcr-Abl (>10 nM), Mast/stem cell growth factor receptor Kit (16 nM). Imatinib is a kinase inhibitor indicated for treatment of adult and pediatric patients with Philadelphia chromosome positive chronic myeloid leukemia (Ph+ CML) and acute lymphoblastic leukemia (Ph+ ALL).

A series of case reports have demonstrated a potential role of kinase inhibitors, such as Imatinib, in regulating vascular permeability. In these studies, treatment with Imatinib was associated with rapid resolution of pulmonary and systemic vascular leak.

In addition to the anti-inflammatory effects, Imatinib may also exhibit antiviral properties. The antiviral activity of Imatinib appears to occur at the early stages of infection, following cellular entry and endosomal trafficking, by inhibiting fusion of the virus at the endosomal membrane. The potential antiviral properties of this drug have been previously demonstrated in preclinical assays where Imatinib demonstrated a potent inhibitory effect on SARS-CoV and MERS-CoV replication in in vitro assays, mainly via its inhibition of ABL type 2 kinase. In these studies, Imatinib demonstrated low toxicity and inhibited SARS-CoV and MERS-CoV with EC50 values in the range of 9.8 to 17.6 μM. These data suggest that the ABL1 pathway may be important for replication of different virus families and, therefore, inhibitors of this pathway have the potential to be broad-spectrum antivirals. Additionally, pharmacokinetic studies have demonstrated that the IC50 of Imatinib for ABL1, BCR-ABL1, and ABL2 kinase inhibition is around 0.3 μM, which is below the expected trough plasma concentration (1.7 μM) of an oral Imatinib dose of 400 mg/day. Initial estimates suggest an EC50 value of approximately 2.5 μM for the inhibition of SARS-CoV-2 virus. This concentration is achievable in vivo following administration of an oral dose of 800 mg/day Imatinib in patients, thus one of the therapeutically effective amounts comprised in the combination therapy according to embodiments of the present disclosure.

According to some embodiments of the present disclosure, Dabrafenib, which is a BRAF V600 inhibitor, may be useful in treating COVID-19 because of its actions as a Multi-kinase inhibitor. Dabrafenib is known to target STK B-raf (0.4 nM), STK A-raf (26 nM), and STK RAF proto-oncogene (150 nM).

According to some embodiments of the present disclosure, Entrectinib, which is a tyrosine kinase inhibitor, may be useful in treating COVID-19 because of its actions as a Multi-kinase inhibitor. Entrectinib is known to target NTRK1 (0.6 nM), BDNF/Nt-3 (3 nM), ROS1 (7 nM), TK ALK (12 nM), TK JAK2 (40 nM), and IGF-1 (122 nM).

According to some embodiments of the present disclosure, Sorafenib, which is a tyrosine kinase inhibitor, may be useful in treating COVID-19 because of its actions as a multi-kinase inhibitor, and/or immunosuppressant and/or ability to inhibit viral replication and protein production. Sorafenib is known to target VEGF2 (0.16 nM), ephrin type-B receptor 4 (0.2 nM), angiopoietin-1 receptor (0.83 nM), c-RAF (1 nM), VEGF3 (3 nM), EGFR (3 nM), fibroblast growth factor receptor 1 (4.60 nM), proto-oncogene TK Ret (6 nM), discoidin domain-containing receptor 2 (7 nM), RAF (7.10 nM), and BRAF V600E (11 nM).

According to embodiments of the present disclosure, Dacomitinib, which is a tyrosine kinase inhibitor, would be useful in treating COVID-19 because of its actions as an EGFR inhibitor to prevent excessive fibrotic response and impact viral entry. Dacomitinib is known to target EGFR (1.80 nM), TK erbB-2 (16.7 nM), TK erbB-4 (74 nM), TK Lck (94 nM), and proto-oncogene TK Src (110 nM).

According to embodiments of the present disclosure, Sunitinib, which is a tyrosine kinase inhibitor, would be useful in treating COVID-19 because of its actions as a Tyrosine kinase inhibitor. Sunitinib is known to target VEGF1 (1 nM), Mast/stem cell growth factor receptor Kit (1.1 nM), Platelet-derived growth factor receptor beta (2 nM), TK FLT3 (3 nM), VEGF2 (5.5 nM), TK Lck (8.9 nM), and VEGF3 (8.9 nM).

According to some embodiments of the present disclosure, Alectinib, which is an ALK and RET inhibitor, may be useful in treating COVID-19 because of its actions as a Tyrosine kinase inhibitor. Alectinib is known to target TK ALK (5.3 nM) and TK Ret (4.8 nM).

According to some embodiments of the present disclosure, Baricitinib, which is a JAK inhibitor, may be useful in treating COVID-19 because of its actions as a Tyrosine kinase inhibitor, JAK inhibitor. Baricitinib is known to target TK JAK1 (0.7 nM), JAK2 (0.8 nM), TYK2 (8.7 nM), JAK1/TYK2 (15 nM), JAK1/JAK2/TYK2 (21 nM), JAK3 (25 nM), JAK2/TYK2 (149 nM), and JAK3/JAK1 (259 nM).

According to some embodiments of the present disclosure, Ibrutinib, which is a Bruton's tyrosine kinase (BTK) inhibitor, may be useful in treating COVID-19 because of its mode of action. Ibrutinib is known to target BTK (0.1 nM), BMX (0.8 nM), EGFR (1.3 nM), TXK (2.3 nM), TEC (1.4 nM), and erbB-4 (0.1 nM).

According to some embodiments of the present disclosure, compounds having displayed a propensity for hindering the entry of coronavirus particles into a mammalian cell are selected from the group consisting of: Mebendazole; Crizotinib; Bosutinib; Vandetanib; Midostaurin; and Alectinib.

According to some embodiments of the present disclosure, compounds having displayed a propensity for hindering the fusion of coronavirus particles with a mammalian cell are selected from the group consisting of: Mebendazole; Bosutinib; Dabrafenib; Sorafenib; and Sunitinib.

According to some embodiments of the present disclosure, compounds having displayed a propensity for hindering the replication of coronavirus particles once inside a mammalian cell are selected from the group consisting of: Niclosamide; and Imatinib.

Based on pre-clinical models, Mebendazole given orally, showed only about 15% bioavailability and the rest of the drug remained in the gastrointestinal tract. On the contrary, Imatinib showed an excellent absorption (98% bioavailability after oral administration). In some embodiments of the present disclosure, the two drugs may be administered with at least 60-minutes between administration. For example, treatment could start with administering a therapeutically effective amount of Imatinib, waiting about 60 minutes or more, followed by administering a therapeutically effective amount of Mebendazole. This approach could minimize any possible drug-drug interactions during treatment.

In some embodiments of the present disclosure, Mebendazole and Imatinib may be used as agents, individually or in combination, with the same or different therapeutically effective amounts. As a non-limiting example, Mebendazole and Imatinib may be provided in one or two or more medicaments that deliver a dose of between 1 mg and 1000 mg of each or both of Mebendazole and Imatinib. In further non-limiting examples, the one or two or more medicaments may deliver a single dose of each or both of Mebendazole and Imatinib of between about 5 mg and about 995 mg, between about 10 mg and about 990 mg, between about 25 mg and about 975 mg, between about 50 mg and 950 mg, between about 75 mg and 925 mg, between about 100 mg and about 900 mg, between about 200 mg and 800 mg, between about 300 mg and 700 mg, between about 500 mg and 600 mg and combinations thereof.

In some embodiments of the present disclosure, Mebendazole may be administered in a daily dose of between about 25 mg and about 500 mg. In some embodiments of the present disclosure, Mebendazole may be administered in a daily dose of between about 50 mg and about 450 mg, about 75 mg and about 400 mg, between about 100 mg and about 350 mg, between about 125 mg and about 300 mg, about 150 mg to about 250 mg, between about 175 mg and about 225 mg and all dose ranges therebetween. In some embodiments of the present disclosure, the daily dose of Mebendazole may be about 200 mg.

In some embodiments of the present disclosure, a target cell may be exposed to a therapeutically effective amount if treated with a concentration of Mebendazole of between about 50 nM and about 2200 nM, between about 75 nM and about 2100 nM, between about 100 nM and about 2000 nM, between about 150 nM and about 1800 nM, between about 200 nM and about 1600 nM, between about 300 nM and about 1400 nM, between about 400 nM and about 1200 nM, between about 500 nM and about 1000 nM, between about 600 nM and about 800 nM and all concentration ranges therebetween

In some embodiments of the present disclosure, Imatinib may be administered in a daily dose of between about 250 mg and 1500 mg. In some embodiments of the present disclosure, Imatinib may be administered in a daily dose of between about 300 mg and about 1400 mg, between about 400 mg and about 1300 mg, between about 500 mg and about 1200, between about 600 mg and about 1100 mg, between about 700 mg and about 1000 mg, between about 800 mg and about 900 mg and all dose ranges therebetween. In some embodiments of the present disclosure, the daily dose of Imatinib may be about 800 mg.

In some embodiments of the present disclosure, a target cell may be exposed to a therapeutically effective amount if treated with a concentration of Imatinib of between about 5 nM and about 120 nM, between about 10 nM and about 100 nM, between about 20 nM and about 90 nM, between about 30 nM and about 80 nM, between about 40 nM and about 70 nM, between about 50 nM and about 60 nM and all concentration ranges therebetween.

Some embodiments of the present disclosure relate to a pharmaceutical composition that comprises both Mebendazole and Imatinib with the same or different therapeutically effective amounts of each agent for treating COVID-19. In some embodiments of the present disclosure, the pharmaceutical composition further comprises one or more carriers and/or one or more excipients. In a non-limiting example of the present disclosure, the pharmaceutical composition comprises Mebendazole and/or Imatinib, at least one monoglycerides and/or at least one diglyceride and/or tocopherol polyethylene glycol succinate. In a further non-limiting example of the present disclosure, the pharmaceutical composition may further comprise a lipid-based carrier, such as an emulsion of micro-sized particles and/or nanoparticles. At least one example of suitable nanoparticles are those comprising poly (lactide-co-glycolide).

### EXAMPLES

The DeepDrug™ computational Artificial Intelligence (AI) system was used to identify various tyrosine kinase inhibitors as likely to be effective against SARS-CoV-2 based on the similarity of the drugs to antiviral peptides (AVPs) known to target SARS-CoV-1 and other viruses. AVPs are fragments of human proteins that respond to a viral infection by targeting key steps in the viral replication life-cycle including (1) virus binding to the cell surface and internalizing into endosomal compartments (entry), (2) virus being released from endosomal compartments into the cytosol (fusion), and (3) viral protein processing and replication of the viral genome (replication) Protein-Protein binding.

An AI technique was used to generate “fingerprints” for various tyrosine kinase inhibitors and the AVPs in a mathematical representation of all protein interactions in a cell (as described by Grover, A. & Leskovec, J. (2016) node2vec: Scalable Feature Learning for Networks. Kdd, 2016, 855-864). This mathematical representation was created based on the following datasets: AVPdb, a dataset of 2,683 AVPs including 98 from SARS-CoV-1; HPIDB, a dataset of 981 HIV AVPs; hu.map, a dataset of 17.5 million protein-protein interactions; Corum, a dataset of 4,274 mammalian protein complexes; STRING, a dataset of 4,584,628 proteins from 5,090 organisms; DrugBank, a dataset of 13,491 drugs; and BindingDB, a dataset of 846,857 drugs and 7,605 proteins.

An AI technique called a Siamese Network (SNet) was used to compare the fingerprints of various tyrosine kinase inhibitors to the fingerprints of AVPs. SNet predictions were based on a small number of SARS-CoV-1 AVPs that exhibited the strongest antiviral effects. Additionally, SNet provided separate predictions for the three mechanisms of viral infection (e.g., entry, fusion, and replication), which afforded a higher degree of specificity in drug screening. The SNet projected fingerprints into a multidimensional space and calculated distances between them, and the closer the prediction was to zero, the more similar a pair of fingerprints were and the more a drug resembled AVPs. Predictions less than the optimal threshold of 0.63 indicate a significant similarity between the fingerprint of a drug and AVP (i.e., a drug having antiviral effects).

Many of the tyrosine kinase inhibitors, including Mebendazole and Imatinib, received significant support (i.e., SNet distances closest to zero) for each of the viral mechanisms (e.g., entry, fusion, and replication). Based on comprehensive analysis of SNet predictions for 4,118 FDA-approved drugs, Mebendazole and Imatinib ranked within the top 99th percentile for each mechanism. Table A below summarizes the SNet prediction data for Mebendazole and Imatinib.

TABLE A
SNet Predictions of Imatinib and Mebendazole
for Entry, Fusion, and Replication.

| | Entry | Fusion | Replication |
|---|---|---|---|
| | Prediction | Prediction | Prediction |
| | [Ranking] | [Ranking] | [Ranking] |
| Drug | (Percentile) | (Percentile) | (Percentile) |
| Imatinib | 0.1455 | 0.0955 | 0.2424 |
| | [20^{th}] | [9^{th}] | [27^{th}] |
| | (99.51) | (99.78) | (99.34) |
| Mebendazole | 0.1072 | 0.0796 | 0.2033 |
| | [4^{th}] | [2^{nd}] | [10^{th}] |
| | (99.90) | (99.95) | (99.76) |

An assessment of the potential of small therapeutics to bind with coronavirus particles was carried out. Using three different mechanism potential binding sites for small molecules, the likelihood of protein-protein binding was determined using an artificial intelligence computing system. Using a template of the crystal structure of an essential SARS-CoV-2 protease, the functional centers of the protease inhibitor-binding pocket were identified.

Antiviral peptides known to inhibit the SARS virus were used as targets. By creating a fingerprint of the AVPs, they were then compared to similarly generated fingerprints (embedding) of individual compounds to identify the ones most closely related.

The AVPs used targeted three specific mechanisms: entry, fusion, and replication. The most effective peptides were specifically filtered out and used to create three separate networks based on each peptide's known mechanism of action. This allowed the identification of drugs with certain specificities based on mechanism.

The three mechanisms are relevant for the following reasons. Entry is extremely important because inhibiting viral entry into the cell would reduce the amount of virus that acts on the cell. Likewise, inhibition of replication is important for reducing the amount of viral load generated and spread to other cells after a cell has been infected. Finally, fusion though technically least relevant is worth noting because not all viral entry happens through the standard mechanism. The virus is capable of fusing directly with the membrane of the cell for infection. Though this happens at about 1/10th the rate of the standard entry mechanism, it is still a mechanism which was desirable to use as a focus to attempt to inhibit.

The fingerprints of these specific peptides were created by using the human proteome and a large graph of the proteins involved in all the processes therein. By then comparing these fingerprints to the drug fingerprints, the identification of drugs with a similar (antiviral) effect on the human proteome as the AVPs was carried out.

First Binding Mechanism

A number of compounds where studied to determine their propensity to bind to coronavirus particles according to a first binding mechanism. The interactions where further evaluated by assessing the likelihood the therapeutic compounds would impact the entry of coronavirus into mammalian cells; the fusion of coronavirus particles with mammalian cells; and ultimately the replication of the coronavirus infected cells. Table 1 summarizes the data obtained in this first round of modeling data analysis.

TABLE 1
Results of Protein-Protein modeling data which mimics a
first mechanism of interaction between COVID-19 and each
one of the proposed therapeutic treatment molecules.

| | Modelling score (>0.25 = favorable scores) | | | | |
|---|---|---|---|---|---|
| | | | | | Entry, Fusion, |
| Drug | Corona | Entry | Fusion | Replication | and/or |
| Axitinib | 0.3823 | 0.0729 | 0.1316 | 0.1085 | — |
| Mebendazole | 0.6323 | 0.3207 | 0.3885 | 0.0847 | Entry & |
| | | | | | Fusion |
| Crizotinib | 0.7076 | 0.2824 | 0.009 | 0.0074 | Entry |
| Bosutinib | 0.7619 | 0.4362 | 0.6597 | 0.1013 | Entry & |
| | | | | | Fusion |
| Vandetanib | 0.463 | 0.4284 | 0.0648 | 0.0172 | Entry |
| Midostaurin | 0.563 | 0.251 | 0.0751 | 0.1797 | Entry |
| Gefitinib | 0.2336 | 0.0451 | 0.0108 | 0.0431 | — |
| Niclosamide | 0.4918 | 0.1698 | 0.0183 | 0.3355 | Replication |
| Imatinib | 0.5696 | 0.0892 | 0.0092 | 0.3522 | Replication |
| Dabrafenib | 0.8273 | 0.1521 | 0.9719 | 0.0753 | Fusion |
| Entrectinib | 0.1849 | 0.0455 | 0.0162 | 0.0089 | — |
| Sorafenib | 0.4455 | 0.0289 | 0.7011 | 0.004 | Fusion |
| Dacomitinib | 0.2218 | 0.0609 | 0.0647 | 0.0272 | — |
| Sunitinib | 0.4571 | 0.0345 | 0.2619 | 0.0087 | Fusion |
| Alectinib | 0.4115 | 0.2454 | 0.0854 | 0.0598 | Entry |
| Baricitinib | 0.2919 | 0.0498 | 0.0973 | 0.1698 | — |
| Ibrutinib | 0.2602 | 0.0285 | 0.036 | 0.0166 | — |

Without being bound to any particular theory, the data collected in the study of the first binding mechanism, the majority of the compounds analyzed demonstrated a theoretical propensity to bind to SARS-CoV-2 viral particles.

Second Binding Mechanism

The same therapeutic compounds were subsequently studied to determine their propensity to bind to SARS-CoV-2 viral particles according to a second binding mechanism. The interactions where also further evaluated by assessing the likelihood the therapeutic compounds would impact the entry of SARS-CoV-2 virus into mammalian cells; the fusion of SARS-CoV-2 viral particles with mammalian cells; and ultimately the replication of the SARS-CoV-2 infected cells. Table 2 summarizes the data obtained in this second round of modeling data analysis.

TABLE 2
Results of Protein-Protein modeling data which mimics a second
mechanism of interaction between the SARS-CoV-2 virus and
each one of the proposed therapeutic treatment molecules.

| | Modelling score (Snet) | | |
|---|---|---|---|
| | (<0.5 = unfavorable scores) | | |
| Drug | Entry | Fusion | Replication |
| Axitinib | 0.8944 | 0.8742 | 0.8469 |
| Mebendazole | 0.8928 | 0.9204 | 0.7967 |
| Crizotinib | 0.8922 | 0.8902 | 0.8123 |
| Bosutinib | 0.8841 | 0.9267 | 0.7818 |
| Vandetanib | 0.8731 | 0.9158 | 0.7733 |
| Midostaurin | 0.8669 | 0.9123 | 0.7702 |
| Gefitinib | 0.8649 | 0.9084 | 0.7689 |
| Niclosamide | 0.8605 | 0.8939 | 0.7756 |
| Imatinib | 0.8545 | 0.9045 | 0.7576 |
| Dabrafenib | 0.8533 | 0.9179 | 0.751 |
| Entrectinib | 0.8493 | 0.8867 | 0.7634 |
| Sorafenib | 0.8444 | 0.8983 | 0.7475 |
| Dacomitinib | 0.8343 | 0.8886 | 0.7366 |
| Sunitinib | 0.8271 | 0.875 | 0.7401 |
| Alectinib | 0.8048 | 0.8591 | 0.7131 |
| Baricitinib | 0.7651 | 0.826 | 0.6718 |
| Ibrutinib | 0.7965 | 0.8638 | 0.6915 |

Without being bound to any particular theory, the data collected in the study of the second binding mechanism indicates all of the compounds (those having a measured score of greater than 0.5) analyzed demonstrated a theoretical propensity to bind to SARS-CoV-2 viral particles.

Third Binding Mechanism

The same therapeutic compounds were again subsequently studied to determine their propensity to bind to SARS-CoV-2 viral particles according to a third binding mechanism. The interactions where also further evaluated by assessing the likelihood the therapeutic compounds would impact the entry of SARS-CoV-2 virus into mammalian cells; the fusion of SARS-CoV-2 viral particles with mammalian cells; and ultimately the replication of the SARS-CoV-2 infected cells. Table 3 summarizes the data obtained in this third round of modeling data analysis.

TABLE 3
Results of Protein-Protein modeling data which mimics a
third mechanism of interaction between COVID-19 and each
one of the proposed therapeutic treatment molecules.

| | Modelling score Cos Sim (higher = better) | | |
|---|---|---|---|
| Drug | Entry | Fusion | Replication |
| Axitinib | 0.557992986 | 0.546848658 | 0.602707223 |
| Mebendazole | 0.725615118 | 0.719017032 | 0.645826892 |
| Crizotinib | 0.655777473 | 0.593846076 | 0.582417831 |
| Bosutinib | 0.504868066 | 0.542036209 | 0.463812872 |
| Vandetanib | 0.577171206 | 0.527997547 | 0.460996584 |
| Midostaurin | 0.496207088 | 0.507069141 | 0.468555625 |
| Gefitinib | 0.578159021 | 0.568961663 | 0.589154308 |
| Niclosamide | 0.34771437 | 0.364198477 | 0.254359226 |
| Imatinib | 0.527941877 | 0.61502544 | 0.486479492 |
| Dabrafenib | 0.611141058 | 0.871812621 | 0.551328541 |
| Entrectinib | 0.493522951 | 0.475610786 | 0.441472678 |
| Sorafenib | 0.485503156 | 0.635097697 | 0.46819151 |
| Dacomitinib | 0.388281501 | 0.411565293 | 0.381958516 |
| Sunitinib | 0.559478444 | 0.759193684 | 0.496610854 |
| Alectinib | 0.391246934 | 0.303073221 | 0.351154533 |
| Baricitinib | 0.328589285 | 0.366532048 | 0.444422964 |
| Ibrutinib | 0.724668227 | 0.639315875 | 0.756544823 |

Without being bound by any particular theory, the data collected in the study of the third binding mechanism indicates all of the compounds analyzed demonstrated a theoretical propensity to bind to SARS-CoV-2 viral particles.

In Vitro Tests

In vitro tests were performed on Vero-76 cells against NR-53516 SARS-Related Coronavirus 2 Isolate New York—PV09158/2020 (New York strain of SARS COV 2), Axitinib showed a reduction in “viral load” (Average Virus TCID50/ml*10\hat{ }6) after 4 days by 66.4% at 500 nano molar concentration, Bosutinib showed a reduction by 47.3% at 50 nano molar, Gefitinib showed a reduction by 53.54% at 50 nano molar, Imatinib showed a 29.6% reduction at 10 micro molar, thereby corroborating the approach taken and the results obtained.

The in vitro efficacy of Mebendazole and other potential antiviral compounds against SARS-CoV-2 (USA-WA1/2020 Isolate) was assessed in human lung cancer (Calu-3) cells. A stock solution of 4 μM of Mebendazole was prepared in DMSO and tested at eight concentrations of 5000 nM, 1670 nM, 555.6 nM, 185.2 nM, 61.7 nM, 20.6 nM, 6.9 nM, and 2.3 nM. Calu-3 cells were cultured in 96-well plates and tested in triplicate. A pretreatment/treatment regimen was utilized where cells were incubated with Mebendazole for 24±4 hours, cells were then inoculated at a multiplicity of infection (MOI) of 0.005 TCID50 (median tissue culture infectious dose) per cell (200 TCID50/well) with SARS-CoV-2 and incubated for 60-90 minutes. Immediately following incubation, viral inoculum was removed, cells were washed, and wells were overlaid with 0.2 mL Eagle's Modified Essential Media (EMEM) with 2% Fetal Bovine Serum (FBS) containing Mebendazole or control articles and incubated in a humidified chamber at 37° C.±2° C. in 5±2% CO2. At 48±6 hours following virus inoculation, cells were fixed and evaluated for the presence of virus by the immunostaining assay (for cytopathic effect (CPE) by the MTT [3-(4,5-dimethylthiazol-2-yl)-2,5-diphenyltetrazolium bromide] assay).

For each well on the assay plate, inhibition of SARS-CoV-2 was calculated as the percentage of reduction of the absorbance value (A450 of Mebendazole dilution) relative to mean absorbance values from wells designated as positive control (A450 virus control; no drug and 0% inhibition) and negative control (A450 of cell control; no virus and 100% inhibition) by the following formula:

$$Percent Inhibition = 100 - ( \frac{A 450 of mebendazole dilution - A 450 of cell control}{A 450 of virus control - A 450 of cell control} ) \times 100$$

The effective concentration (EC50) was defined as the concentration of Mebendazole that causes 50% reduction of the mean absorbance value of the virus control (0% inhibition) relative to the cell control (100% inhibition). The cytotoxicity of Mebendazole at the 8 concentrations was also evaluated as determined by percent inhibition of cell viability.

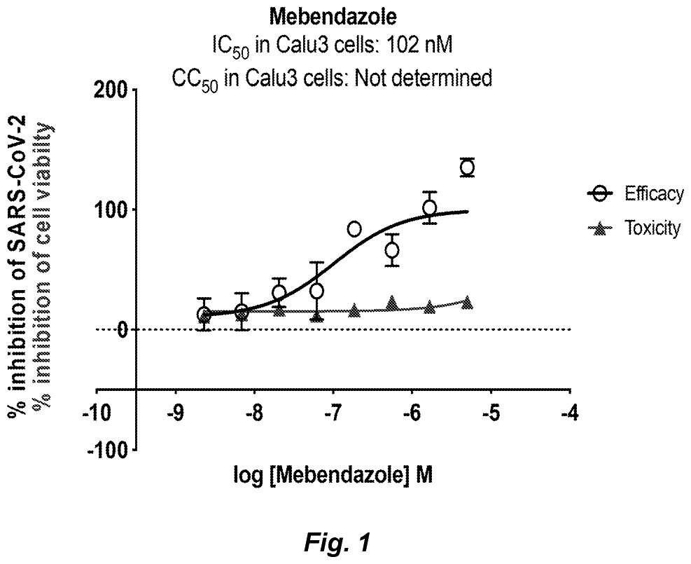

Complete or near complete inhibition of SARS-CoV-2 with an EC50 of 102 nM was determined for Mebendazole (see FIG. 1). The cytotoxic concentration (CC50) was estimated at >5 μM based on an absence of observed cytotoxicity (˜0% inhibition of cell viability) at all tested concentrations up to a maximum concentration of 5000 nM, indicating a wide margin of safety between the effective concentrations and the cytotoxic concentrations reported in these human lung cells.

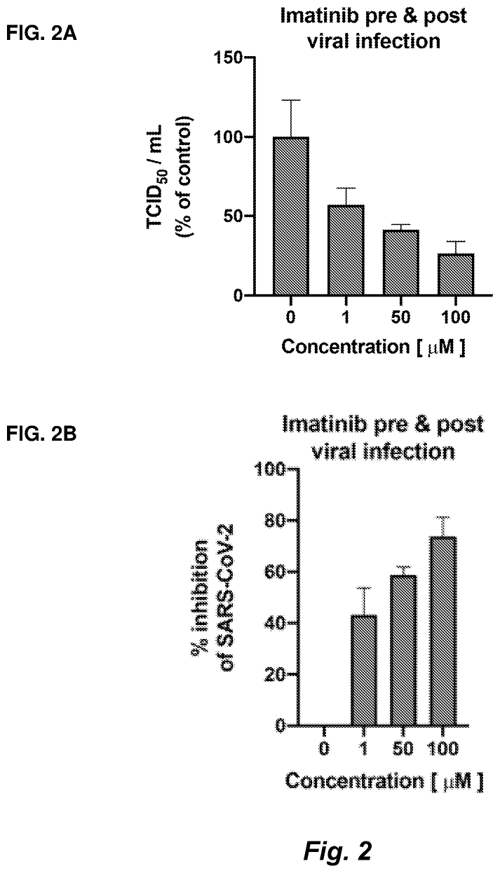

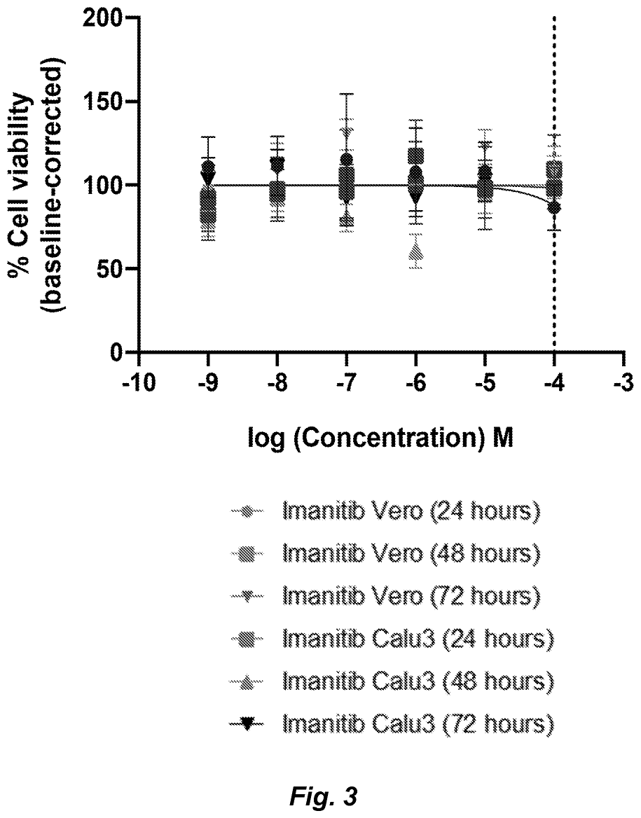

Imatinib was also examined in by in vitro experiments. Briefly, Vero 76 African green monkey kidney cells were infected with SARS-CoV-2 (New York-PV091158/2020 strain). Imatinib was added to cells at the same time as the virus (“Imatinib pre & post viral infection”), or after a 1-hour incubation with the virus (“Imatinib post viral infection”) in order to evaluate if the presence of drug could impair entry of the virus into the cell, which could be represented in the data as increased SARS-CoV-2 inhibition. For the pre- & post-treatment experiment, higher concentration of Imatinib demonstrated a lower TCID50/ml (% of control) (see FIG. 2A). The percent inhibition of SARS-CoV-2 by Imatinib was 43% (1 μM), 51% (50 μM), and 74% (100 μM) (see FIG. 2B). For the post-treatment arm, SARS-CoV-2 inhibition by Imatinib was 24% (1 μM), 3% (50 μM), and 55% (100 μM) (data not shown). For both arms, SARS-CoV-2 inhibition by the highest concentration of Imatinib (100 μM). Cytotoxicity experiments were performed, using the MTT cell proliferation assay, in parallel using the same experimental conditions and including another cell line (Calu-3) in addition to Vero 76 cells, and no cytotoxicity was observed at any concentration tested (up to 100 μM) (see FIG. 3). Without being bound by any particular theory, these data show that Imatinib appreciably inhibited SARS-CoV-2 at concentrations as low as 1 μM and was not cytotoxic at 100 μM, lending support for a good therapeutic index in humans. Furthermore, increased antiviral activity was observed when Imatinib was added to cells prior to the addition of the virus to the cells, suggesting that Imatinib may be affecting how the virus enters cells in addition to affecting viral replication.

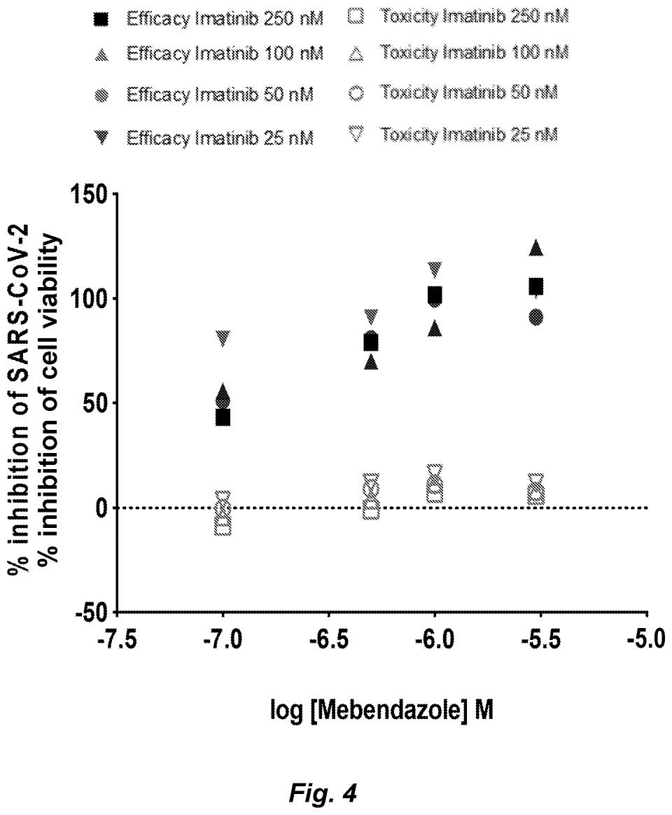

The in vitro efficacy and cytotoxicity of Imatinib in combination with Mebendazole was evaluated in Calu-3 cells (see FIG. 4). Imatinib at 4 different concentrations (25, 50, 100, or 250 nM) and Mebendazole at 4 concentrations (0.1, 0.3, 1, and 3 μM) were added to cells following the same protocol as described above for FIG. 1. Imatinib in combination with Mebendazole at these drug concentrations achieved dose-dependent inhibition of SARS-CoV-2 without clear evidence of cytotoxicity.

Further Experimental Tests

The objective of the in vivo study was to test the in vivo efficacy of antiviral therapeutics against SARS-CoV-2, the causative agent of COVID-19, in the ACE2 mouse model.

In an animal biosafety level-3 suite, all animal procedures were performed in accordance with the protocols approved by the animal care and use committee at a contract research organization (CRO) in the United States. Study animals were randomized and divided into 6 groups. Prior to viral challenge, mice were monitored for 3 days to establish baseline body weight. Mice were then challenged with the USA-WA1/2020 (SARS-CoV-2) virus on Study Day 0. Mice were treated via oral gavage starting 4-6 hours after viral challenge then once daily on Study Days 1-10 at 24±2 hours post viral challenge. Mouse body weight, and survival were monitored for 14 days after challenge. On day 6, blood was collected for clinical pathology and organs harvested for viral titers and histopathology from 7 mice in each group. On day 14, blood was collected for clinical pathology and organs harvested for histopathology from 4 mice in each group.

A cohort of 66 female K18-hACE2 (B6.Cg-Tg(K18-ACE2)2PrlmnJ) mice were purchased from Jackson Labs (Bar Harbor, ME) for use in the in vivo study. The animals were approximately 7-10 weeks of age upon study initiation and weighed approximately 16-20 grams.

The animals purchased for use in this study were held in quarantine for a minimum of 7 days prior to assignment to the study. During the quarantine period, animals were observed at least once daily. At the conclusion of the quarantine period, the health status of the animals was reviewed by a Staff Veterinarian and only healthy animals were released for testing.

Mice were assigned to groups using a computerized randomization procedure based on body weights that will produce similar group mean values [ToxData® version 3.0 (PDS Pathology Data Systems, Inc., Basel, Switzerland)]. No animal's body weight varied from the mean of the group body weight by more than 20%. The groups were organized as follows: G1—untreated control group; G2—received 100 mg/kg of Imatinib; G3—received 200 mg/kg of Niclosamide; G4—received Imatinib (100 mg/kg) and Niclosamide (200 mg/kg); G5—received 50 mg/kg of Mebendazole; and, G6—received Imatinib (100 mg/kg) and Mebendazole (50 mg/kg).

Animal rooms were lighted with fluorescent lights and maintained on a 12-hr light/dark cycle. To the maximum extent possible, room temperature was maintained at approximately 20-26° C. and relative humidity at approximately 30-70% in accordance with the National Research Council's “Guide for the Care and Use of Laboratory Animals”, 2011. Room temperature and relative humidity values were recorded daily.

Administration of Test and Control Articles

From Study Day 0 through Study Day 10, mice were administered placebo or test article by oral gavage once daily. The dose on Day 0 took place 4-6 hours post challenge. The doses on Days 1-10 were administered 24±2 hour post viral inoculation and each dose from Day 1-10 were separated by 24±2 hour.

The USA-WA1/2020 (SARS-CoV-2) virus was stored at approximately ≤−65° C. prior to use. For inoculation, mice were anesthetized with a ketamine (100 mg/kg) and xylazine (10 mg/kg) mixture. Once the mouse was anesthetized, it was held such that the nose was pointing upward. About 0.030 ml of inoculum was delivered dropwise into the mouse's nose. The mouse was held upright to allow the virus to be inhaled thoroughly then the mouse was returned to its cage. A quantitative viral infectivity assay (e.g., TCID50 assay) was performed at on a portion of the prepared viral challenge solution.

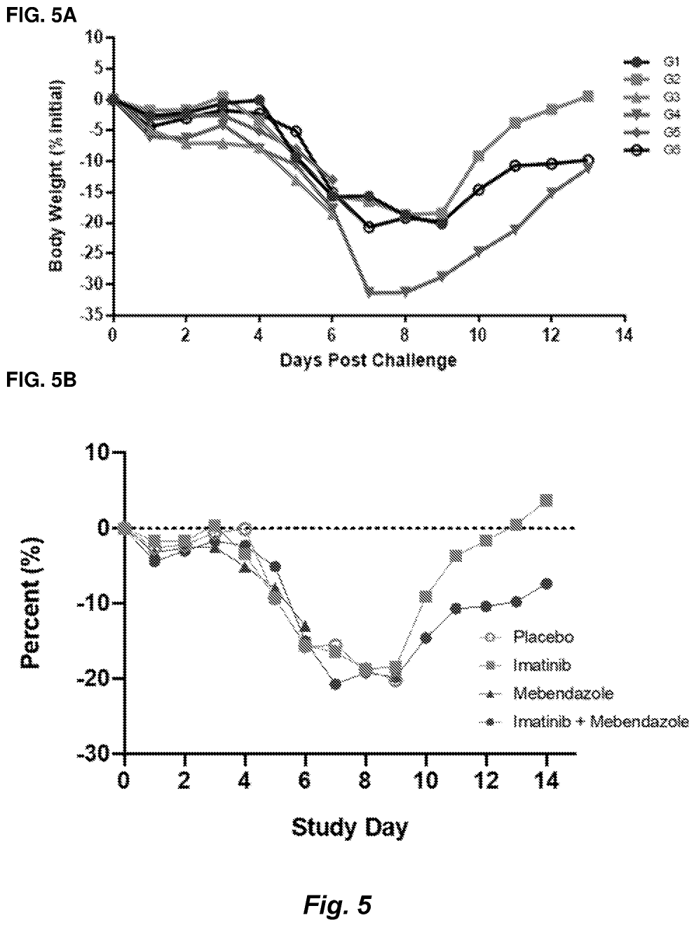

Body weights were recorded within two days of receipt and at randomization. All study animals were weighed prior to challenge then for 14 days post challenge. The change in percent change body weight was calculated for each animal. FIG. 5A shows the body weight data (% of initial body weight) for all of the experimental groups (FIG. 5B shows the body weight data for the placebo group, Imatinib, Mebendazole and Imatinib combined with Mebendazole). Without being bound by any particular theory, it is noted that mice in experimental group G6 (Mebendazole and Imatinib) recovered body weight after the seven day mark.

The necropsy for viral titers took place on Study day 6. Gross pathology examination of the lungs was performed. The lungs (left lung), were harvested from 7 study animals per treatment group on Study Day 6 for viral titers. Tissues for viral titers were weighed then flash frozen in an ethanol/dry ice bath or liquid nitrogen and stored at ≤−65° C. for virus titration and quantitative reverse transcriptase polymerase chain reaction (qRT-PCR, which may also be referred to herein as RT-qPCR) assay at a later date.

The necropsy for histopathology took place on Study Day 6 (n=7/group) and 14 (n=4/group). Gross pathology examination of the lungs was performed. The right lung lobe was inflated by intratracheal infusion of 10% neutral buffered formalin (NBF) then immersed in 10% NBF for fixation. The nasal turbinate, GI-tract (stomach, jejunum, ileum, colon), leg bone and bronchial lymph nodes were collected and immersed in 10% NBF for fixation. All tissues were evaluated with standard hematoxylin and eosin (H & E) staining.

Fixed tissues were processed through to paraffin blocks, sectioned at approximately 5-microns thickness, and stained H & E. All paraffin H & E slides were evaluated microscopically and graded for presence and severity of pathology by a board certified (ACVP) veterinary pathologist.

Blood samples for clinical pathology assays were collected from all study animals on Study Day 6 (n=7/group) and Day 14 (n=4/group). Terminal blood samples was collected from each study mouse vial the retro orbital sinus and processed for serum. The following clinical pathology tests were performed:

1) Serum Chemistry Parameters (AU480
Chemistry System, Beckman Coultier)

| | | |
|---|---|---|
| Sodium | Alkaline phosphatase | Total protein |
| Potassium | Alanine aminotransferase | Total bilirubin |
| | (ALT) | |
| Chloride | Aspartate | Creatinine |
| | aminotransferase (AST) | |
| Calcium | Albumin (A) | Glucose |
| Inorganic phosphorus | Globulin (G) (calculated) | Cholesterol |
| Blood Urea nitrogen | A/G ratio (calculated) | Triglycerides |
| (BUN) | | |
| Creatine kinase (total) | Lactate Dehydrogenase | Gamma-glutamyl |
| | | transferase |

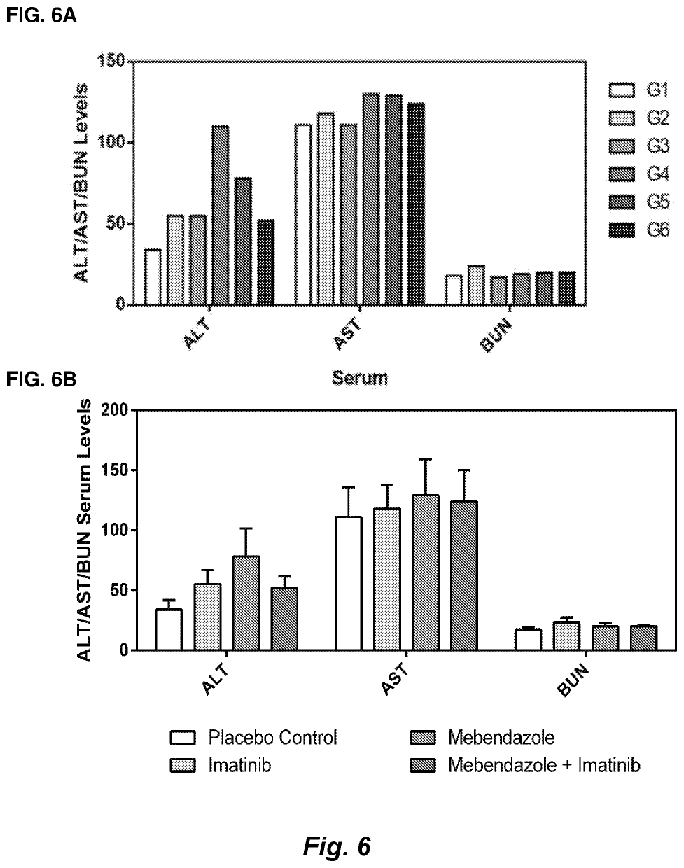

FIG. 6A shows the data obtained for assessing liver and kidney function (AST, ALT and BUN) for all of the experimental groups (FIG. 6B shows the body weight data for the placebo group, Imatinib, Mebendazole and Imatinib combined with Mebendazole). Without being bound by any particular theory, it is noted that experimental group G6 (Mebendazole and Imatinib) did not demonstrate substantially different levels of ALT or BUN as compared to the groups that received Mebendazole (G5) or Imatinib (G2) alone. The mice in experimental group G6 also did not demonstrate substantially different levels of ALT as compared to the group that received Imatinib alone (G2).

Lung RT-qPCR Data for SARS-COV-2 Virus following 6 days of treatment in female K18-hACE2 (B6.Cg-Tg(K18-ACE2)2PrlmnJ) mice administered either Imatinib 100 mg/kg po (G2), Niclosamide 200 mg/kg po (G3), Mebendazole 50 mg/kg po (G5) and combinations of Imatinib+Niclosamide po (G4) or Imatinib+Mebendazole po (G6) once daily for 6 consecutive days.

The concentration of virus in lung tissue was determined by RT-qPCR assay. Briefly, RNA was extracted from samples stored in RNA/DNA Shield using the Quick-RNA Viral Kit (Zymo Research) according to manufacturer's protocol. RT-qPCR was performed using the following RT-PCR cycling conditions: 50° C. for 15 min (RT), then 95° C. for 2 min (denature), then 40 cycles of 10 s at 95° C., 45 s at 62° C. Virus titer by RT-qPCR was performed according to the CRO's standard operating procedures.

Primers Used for SARS-CoV-2 Detection:

SEQ ID No. 4 (2019-nCoV_N1-F):
5′-GACCCCAAAATCAGCGAAAT-3′
SEQ ID No. 5 (2019-nCoV_N1-R):
5′-TCTGGTTACTGCCAGTTGAATCTG-3′
SEQ ID No. 6 (Probe: 2019-nCoV_N1-P):
5′-FAM-ACCCCGCATTACGTTTGGTGGACC-BHQ1-3′

TABLE 4
Mean Viral Content assessed by Lung RT-qPCR

| G1 | | | G2 | | | G3 | | |
|---|---|---|---|---|---|---|---|---|
| | Mean | | | Mean | | | Mean | |
| An# | Result | Log₁₀ | An# | Result | Log₁₀ | An# | Result | Log₁₀ |
| 01 | 4.86E+07 | 7.7 | 13 | 7.53E+07 | 7.9 | 25 | 7.72E+07 | 7.9 |
| 02 | 6.40E+07 | 7.8 | 14 | 3.12E+08 | 8.5 | 26 | 2.97E+07 | 7.5 |
| 03 | 6.67E+07 | 7.8 | 15 | 3.67E+07 | 7.6 | 27 | 4.28E+07 | 7.6 |
| 04 | 2.54E+06 | 6.4 | 16 | 5.37E+07 | 7.7 | 28 | 5.54E+06 | 6.7 |
| 05 | 8.70E+07 | 7.9 | 17 | 7.80E+07 | 7.9 | 29 | 3.04E+07 | 7.5 |
| 06 | 2.13E+08 | 8.3 | 18 | 9.13E+07 | 8.0 | 30 | 9.85E+07 | 8.0 |
| 07 | 6.51E+07 | 7.8 | 19 | 2.15E+07 | 7.3 | 32 | 3.08E+08 | 8.5 |
| AVG | 7.81E+07 | 7.7 | AVG | 9.54E+07 | 7.8 | AVG | 8.46E+07 | 7.7 |
| G4 | | | G5 | | | G6 | | |
| | Mean | | | Mean | | | Mean | |
| An# | Result | Log₁₀ | An# | Result | Log₁₀ | An# | Result | Log₁₀ |
| 34 | 3.94E+06 | 6.6 | 45 | 7.15E+07 | 7.9 | 56 | 2.41E+07 | 7.4 |
| 35 | 6.15E+07 | 7.8 | 48 | 4.97E+07 | 7.7 | 58 | 1.94E+07 | 7.3 |
| 36 | 8.26E+07 | 7.9 | 49 | 7.76E+07 | 7.9 | 59 | 1.51E+08 | 8.2 |
| 38 | 1.19E+07 | 7.1 | 50 | 1.90E+07 | 7.3 | 60 | 3.92E+07 | 7.6 |
| 39 | 8.13E+07 | 7.9 | 52 | 2.03E+07 | 7.3 | 61 | 7.66E+07 | 7.9 |
| 40 | 1.02E+08 | 8.0 | 54 | 3.73E+07 | 7.6 | 62 | 2.36E+06 | 6.4 |
| 41 | 8.29E+07 | 7.9 | 55 | 2.95E+07 | 7.5 | 63 | 2.11E+06 | 6.3 |
| AVG | 6.09E+07 | 7.6 | AVG | 4.36E+07 | 7.8 | AVG | 4.50E+07 | 7.3 |

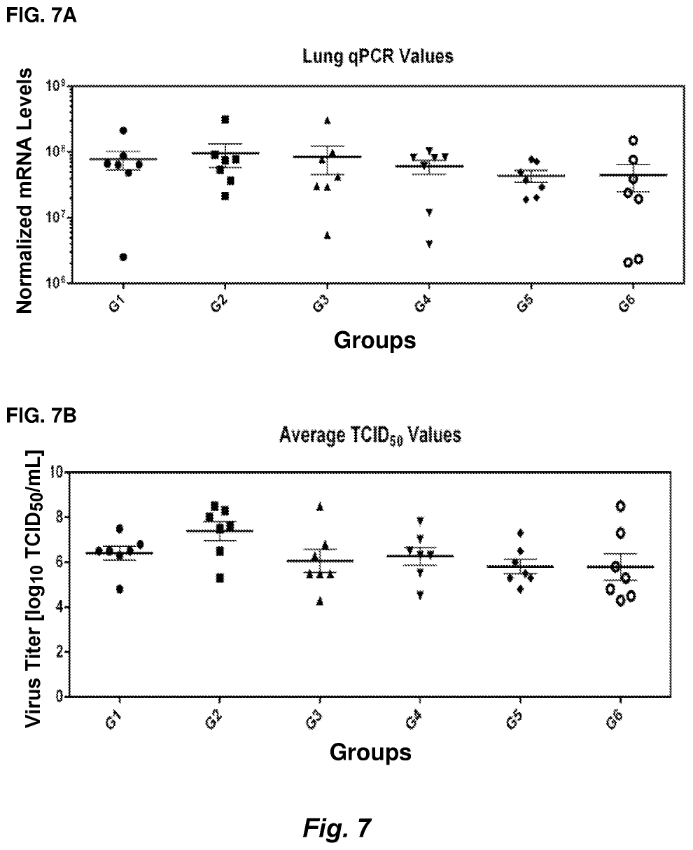

FIG. 7A shows normalized mRNA levels and FIG. 7B shows the virus titer data obtained by the lung RT-qPCR analysis performed on samples from all experimental groups.

TABLE 5
Individual data LUNG TCID₅₀ Data

| | Animal | Lung (log₁₀ | | Animal | Lung (log₁₀ | | Animal | Lung (log₁₀ |
|---|---|---|---|---|---|---|---|---|
| Group | Number | TCID₅₀/mL) | Group | Number | TCID₅₀/mL) | Group | Number | TCID₅₀/mL) |
| 1 | 01 | 6.5 | 2 | 13 | 7.6 | 3 | 25 | 6.8 |
| | 02 | 6.5 | | 14 | 8.5 | | 26 | 5.5 |
| | 03 | 6.8 | | 15 | 8.0 | | 27 | 5.5 |
| | 04 | 4.8 | | 16 | 6.5 | | 28 | 4.3 |
| | 05 | 6.5 | | 17 | 8.3 | | 29 | 6.3 |
| | 06 | 7.5 | | 18 | 7.5 | | 30 | 5.5 |
| | 07 | 6.3 | | 19 | 5.3 | | 32 | 8.5 |
| | AVG | 6.4 | | AVG | 7.4 | | AVG | 6.1 |
| | SD | 0.8 | | SD | 1.1 | | SD | 1.3 |
| 4 | 34 | 6.5 | 5 | 45 | 6.0 | 6 | 56 | 5.3 |
| | 35 | 6.3 | | 48 | 5.3 | | 58 | 7.3 |
| | 36 | 7.0 | | 49 | 4.8 | | 59 | 4.8 |
| | 38 | 4.5 | | 50 | 5.5 | | 60 | 5.8 |
| | 39 | 5.5 | | 52 | 5.3 | | 61 | 8.5 |
| | 40 | 6.3 | | 54 | 7.3 | | 62 | 4.3 |
| | 41 | 7.8 | | 55 | 6.5 | | 63 | 4.5 |
| | AVG | 6.3 | | AVG | 5.8 | | AVG | 5.7 |
| | SD | 1.1 | | SD | 0.9 | | SD | 1.6 |

“AVG” = average;
“SD” = Standard Deviation;

In reviewing the individual data in the above table, there may be a significant impact from the administration of Mebendazole on mice which may indicate a positive response to the drug. In fact, when reviewing the results of mice #59, 60, 62 and 63 a significant difference from the average of the placebo group (group 1). In group 5, two mice can be said to have responded negatively to the administration of Mebendazole while a single one did not seem have had been significantly affected either positively or negatively.

Interestingly, the mice in Group 6 (combination treatment of Imatinib and Mebendazole) had a potentiated effect compared to the Group 2 (Imatinib only) and Group 5 (Mebendazole only). A positive impact was observed in more animals (5 out of 7) while the remaining animals were negatively impacted by the administration of the drug combination. Recall, in Group 2 a single animal was significantly positively affected by the administration of Imatinib, while 4 were negatively affected and 2 animals were seemingly not significantly affected.

Table 6 shows the percent reduction of lung tissue from individual mice within the Mebendazole group and the Imatinib combined with Mebendazole group.

TABLE 6
Percent Reduction of Virus in Lung (RT-qPCR Data).

| | Percent Reduction of Lung Tissue |
|---|---|
| | RT-qPCR Titers from Mebendazole |
| | Treated Mice (Individual Animals) |
| | vs. the Group Mean of Placebo |
| Animal Number | Treated Mice |
| 45 | 8.45% |
| 48 | 36.36% |
| 49 | 0.64% |
| 50 | 75.67% |
| 52 | 74.01% |
| 54 | 52.24% |
| 55 | 62.23% |
| Mean % Reduction | 44.2% |
| | Percent Reduction of Lung Tissue |
| | RT-qPCR Titers from Mebendazole + |
| | Imatinib Treated Mice |
| | (Individual Animals) vs. the Group |
| Animal Number | Mean of Placebo Treated Mice |
| 56 | 69.1% |
| 58 | 75.2% |
| 59 | ND |
| 60 | 49.8% |
| 61 | 1.90% |
| 62 | 97.0% |
| 63 | 97.3% |
| Mean % Reduction | 42.4% |

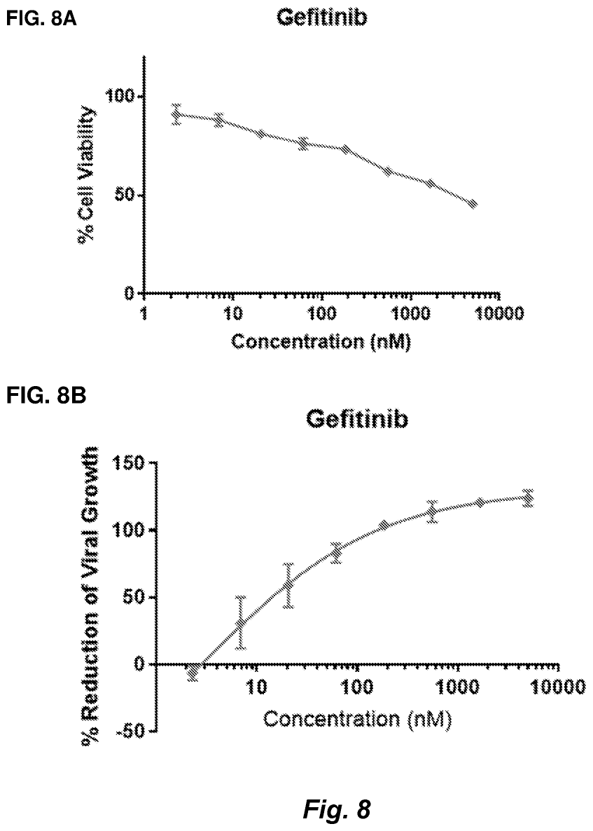

FIG. 8A shows in vitro data from samples, obtained after treating the cells with Gefitinib at various concentrations and FIG. 8B shows the reduction of viral growth data following treatment with Gefitinib. where this data shows cell viability for various concentrations of another tyrosine kinase inhibitor; and FIG. 8B shows reduction in viral growth for various concentrations of the same tyrosine kinase inhibitor of FIG. 8A.

1. A method of treating a subject exposed to a coronavirus, the method comprising steps of:
(a) providing a therapeutically effective amount of a tyrosine kinase inhibitor; and
(b) administering the therapeutically effect amount to the subject,
wherein the treating comprises ameliorating and/or inhibiting some or substantially all of the symptoms of a coronavirus-related disease in the subject.

2. The method of claim 1, further comprising a step of providing a second therapeutically effective amount of a second tyrosine kinase inhibitor and a step of administering the second therapeutically effective amount to the subject.

3. The method of claim 1, wherein the tyrosine kinase inhibitor is one of Axitinib; Mebendazole; Crizotinib; Bosutinib; Vandetanib; Midostaurin; Gefitinib; Niclosamide; Imatinib; Dabrafenib; Entrectinib; Sorafenib; Dacomitinib; Sunitinib; Alectinib; Baricitinib; or Ibrutinib.

4. The method of claim 2, wherein the second tyrosine kinase inhibitor is one of Axitinib; Mebendazole; Crizotinib; Bosutinib; Vandetanib; Midostaurin; Gefitinib; Niclosamide; Imatinib; Dabrafenib; Entrectinib; Sorafenib; Dacomitinib; Sunitinib; Alectinib; Baricitinib; or Ibrutinib.

5. The method of claim 1, wherein the tyrosine kinase inhibitor is Mebendazole.

6. The method of claim 5, wherein the therapeutically effective amount is between about 25 mg and about 500 mg per day.

7. The method of claim 5, wherein the therapeutically effective amount is between about 175 mg and about 225 mg per day.

8. The method of claim 1, wherein the tyrosine kinase inhibitor is Imatinib.

9. The method of claim 8, wherein the therapeutically effective amount is between about 500 mg and about 1200 mg per day.

10. The method of claim 8, wherein the therapeutically effective amount is between about 700 mg and about 1000 mg per day.

11. The method of claim 2, wherein the tyrosine kinase inhibitor is Mebendazole and the second tyrosine kinase inhibitor is Imatinib.

12. The method of claim 11, wherein the effective amount is between about 25 mg and about 500 mg per day and the second effective amount is between about 500 mg and about 1100 mg per day.

13. The method of claim 1, wherein the coronavirus is SARS-CoV-2 or a variant thereof.

14. A method of forming a target cell/agent complex, the method comprising steps of:
(c) administering the therapeutically effect amount of the agent to the target cell; and
(d) forming the target cell/agent complex,
wherein the target cell/complex inhibits fusion of a coronavirus with the target cell, inhibits entry of the coronavirus into the target cell and/or inhibits replication of the coronavirus within the target cell.

15. The method of claim 14, wherein the agent comprises a tyrosine kinase inhibitor.

16. The method of claim 15, wherein the tyrosine kinase inhibitor is one of Axitinib; Mebendazole; Crizotinib; Bosutinib; Vandetanib; Midostaurin; Gefitinib; Niclosamide; Imatinib; Dabrafenib; Entrectinib; Sorafenib; Dacomitinib; Sunitinib; Alectinib; Baricitinib; or Ibrutinib.

17. The method of claim 14, wherein the agent comprises Mebendazole.

18. The method of claim 17, wherein the therapeutically effective amount is between about 75 nM to about 2100 nM.

19. The method of claim 17, wherein the therapeutically effective amount is between about 100 nM and 2000 nM.

20. The method of claim 14, further comprising a step of administering a second therapeutically effect amount of a second agent to the target cell and a step of forming a second target cell/complex.

21. The method of claim 20, wherein the second agent is a second tyrosine kinase inhibitor.

22. The method of claim 21, wherein the second tyrosine kinase inhibitor is one of Axitinib; Mebendazole; Crizotinib; Bosutinib; Vandetanib; Midostaurin; Gefitinib; Niclosamide; Imatinib; Dabrafenib; Entrectinib; Sorafenib; Dacomitinib; Sunitinib; Alectinib; Baricitinib; or Ibrutinib.

23. The method of claim 20, wherein the second agent comprises Imatinib.

24. The method of claim 23, wherein the second therapeutically effect amount is between about 5 nM to about 120 nM.

25. The method of claim 23, wherein the second therapeutically effective amount is between about 10 nM and 100 nM.

26. The method of claim 14, wherein the coronavirus is SARS-CoV-2 or a variant thereof.

27. The method of claim 14, wherein the target cell is an epithelial cell of the upper airways and conducting airways (ciliated and non-ciliated); an alveolar epithelial cell (either type 1 or 2); an epithelial cell of the olfactory system, a neuron of the olfactory system; a neuron of the central nervous system, a neuron of the peripheral nervous system; an epithelial cell of the gastrointestinal tract, an enteroctyte of the gastrointestinal tract, a gland cell of the gastrointestinal tract; a white blood cell, a red blood cell, a thrombocyte, an immune effector cell; a cardiovascular cell, and a renal cell.

28. Use of a tyrosine kinase inhibitor for treating a subject exposed to a coronavirus or infected with a coronavirus.

29. The use of claim 28, further comprising use of a second tyrosine kinase inhibitor.

30. The use of claim 28, wherein the tyrosine kinase inhibitor is one of Axitinib; Mebendazole; Crizotinib; Bosutinib; Vandetanib; Midostaurin; Gefitinib; Niclosamide; Imatinib; Dabrafenib; Entrectinib; Sorafenib; Dacomitinib; Sunitinib; Alectinib; Baricitinib; or Ibrutinib.

31. The use of claim 29, wherein the tyrosine kinase inhibitor and the second tyrosine kinase inhibitor are each one of: one of Axitinib; Mebendazole; Crizotinib; Bosutinib; Vandetanib; Midostaurin; Gefitinib; Niclosamide; Imatinib; Dabrafenib; Entrectinib; Sorafenib; Dacomitinib; Sunitinib; Alectinib; Baricitinib; or Ibrutinib.

32. The use of claim 28, wherein the tyrosine kinase inhibitor is Mebendazole.

33. The use of claim 29, wherein the tyrosine kinase inhibitor is Mebendazole and the second tyrosine kinase is Imatinib.

34. The use of claim 28, the coronavirus is SARS-CoV-2 or a variant thereof.


SEQUENCE LISTING (3 sequences)

SEQ ID NO: 1 - DNA, 20 bp, Artificial Sequence
  Synthetic
gaccccaaaatcagcgaaat

SEQ ID NO: 2 - DNA, 24 bp, Artificial Sequence
  Synthetic
tctggttactgccagttgaatctg

SEQ ID NO: 3 - DNA, 24 bp, Artificial Sequence
  Synthetic
  misc_feature (1)..(1): a is covalently coupled to the fluorescent dye Fluorescein Amadite (FAM)
  misc_feature (24)..(24): c is covalently coupled to the non-fluorescent chromophore Black Hole Quencher-1 (BHQ1)
accccgcattacgtttggtggacc